(19) United States
(12) Patent Application Publication
(10) Pub. No.: US 2025/0110057 A1
(43) Pub. Date: Apr. 3, 2025

(54) HIGH SENSITIVITY BIOLAYER INTERFEROMETRY WITH ENZYME AMPLIFICATION

(71) Applicant: Access Medical Systems, Ltd., Palo Alto, CA (US)

(72) Inventors: Robert F. Zuk, Menlo Park, CA (US); Katie Trieu, Gilroy, CA (US); Alex Ho Fai Lee, Fremont, CA (US); Ruchita Patel, Fremont, CA (US)

(21) Appl. No.: 18/917,939

(22) Filed: Oct. 16, 2024

Related U.S. Application Data
(63) Continuation of application No. PCT/US2023/065858, filed on Apr. 17, 2023.
(60) Provisional application No. 63/363,149, filed on Apr. 18, 2022.

(51) Int. Cl. G01N 21/77; C12Q 1/28; G01N 33/543; G01N 33/58

(52) U.S. Cl. CPC G01N 21/7703; C12Q 1/28; G01N 33/54373; G01N 33/58; G01N 2021/7779; G01N 2333/908

(57) ABSTRACT

The present invention provides a high sensitivity immunoassay method for determining the concentration of an analyte in a sample based on interferometry. The present invention provides a high molecular weight conjugate comprising multiple streptavidins and horse radish peroxidases (HRP) and uses this conjugate to increase the sensitivity of a biochemical assay. The high molecular weight conjugate comprises at least 2 HRPs, preferably 4 HRPs and binds a precipitating HRP substrate to a solid phase to increase the signal of a biochemical assay.

### CROSS-REFERENCE TO RELATED APPLICATION(S)

This application is a continuation of PCT/US2023/065858, filed Apr. 17, 2023; which claim priority to U.S. Provisional Application No. 63/363,149, filed Apr. 18, 2022. The above identified applications are incorporated herein by reference in their entireties.

### FIELD OF THE INVENTION

The present invention related to high sensitivity biochemical assay based on interferometry. The present invention provides a high molecular weight conjugate comprising streptavidin and horse radish peroxidase, and uses this conjugate to increase the sensitivity of biolayer interferometry assay.

### BACKGROUND OF THE INVENTION

BioLayer Interferometry (BLI) is a commonly used analytical method in life science research. The method detects the shift in the optical interference pattern due to the change in the thickness of an optical layer at the tip of a glass probe. The thickness of the optical layer can be altered when a biomolecule binds to the surface of the optical layer. The major applications of the method are characterization of ligand/receptor binding kinetics and biomolecule quantification. However, quantification BLI assays have less sensitivity compared to other methods such as ELISA. BLI assays are also prone to interference by crude biological samples such as cell lysates and blood since any sample non-specific binding to the optical layer can produce a change in the BLI interference signal. The overall application of BLI consequently is limited by the lack of high sensitivity quantification assays and tolerance to crude samples.

### BRIEF DESCRIPTION OF THE DRAWINGS

FIGS. 1A-1C illustrate one example of a biosensor interferometer system.

FIG. 2A shows a spectral interference pattern generated by two light signals reflected from boundaries between first and second refracting surfaces. FIG. 2B shows the spectral interference pattern shifting from T0 to T1, when analyte molecules bind to the analyte-binding molecules on the distal surface of the interference layer.

FIG. 3 depicts one embodiment of a high sensitivity BLI immunoassay for procalcitonin (PCT).

FIG. 4 depicts the flow chart of preparing crosslinked FICOLL® (copolymers of sucrose and epichlorohydrin).

FIG. 5 depicts the flow chart of preparing streptavidin/HRP-Crosslinked FICOLL®.

FIG. 6 depicts the flow chart of preparing anti-fluorescein-crosslinked FICOLL®.

FIG. 7 presents a table of the protocol, reagents, timing steps, volumes, and orbital rpm in performing the procalcitonin (PCT) assay.

FIG. 8 shows the results of the BLI measurements at step 13 monitoring the probe bound HRP enzyme activity when the probe tip is immersed in the substrate reagent.

FIG. 9 shows PCT assay results using streptavidin/HRP crosslinked (Cx) FICOLL® conjugate and streptavidin/HRP conjugate (200,000 Daltons).

FIG. 10 shows results comparing the streptavidin/HR-Cx FICOLL® with two commercial streptavidin-HRP reagents in the PCT assay at 2 ng/ml of PCT.

FIG. 11 shows correlation of clinical PCT samples analyzed by a high sensitivity BLI assay of the present invention and by a fluorescent assay with Pylon 3D instrument (ET Healthcare Inc).

### DETAILED DESCRIPTION OF THE INVENTION

### Definitions

Terms used in the claims and specification are to be construed in accordance with their usual meaning as understood by one skilled in the art except and as defined as set forth below.

“About,” as used herein, refers to within ±10% of the recited value.

An “analyte-binding” molecule, as used herein, refers to any molecule capable of participating in a specific binding reaction with an analyte molecule. Examples include but are not limited to, (i) antigen molecules, for use in detecting the presence of antibodies specific against that antigen; (ii) antibody molecules, for use in detecting the presence of antigens; (iii) protein molecules, for use in detecting the presence of a binding partner for that protein; (iv) ligands, for use in detecting the presence of a binding partner; or (v) single stranded nucleic acid molecules, for detecting the presence of nucleic acid binding molecules.

An “aspect ratio” of a shape refers to the ratio of its longer dimension to its shorter dimension.

A “binding molecule,” refers to a molecule that is capable to bind another molecule of interest.

“A binding pair,” as used herein, refers to two molecules that are attracted to each other and specifically bind to each other. Examples of binding pairs include, but not limited to, an antigen and an antibody against the antigen, a ligand and its receptor, complementary strands of nucleic acids, biotin and avidin, biotin and streptavidin, lectin and carbohydrates. Preferred binding pairs are biotin and streptavidin, biotin and avidin, fluorescein and anti-fluorescein, digioxigenin/anti-digioxigenin.

“Immobilized,” as used herein, refers to reagents being fixed to a solid surface. When a reagent is immobilized to a solid surface, it is either be non-covalently bound or covalently bound to the surface.

“A monolithic substrate,” as used herein, refers to a single piece of a solid material such as glass, quartz, or plastic that has one refractive index.

A “probe,” as used herein, refers to a monolithic substrate having as aspect ratio (length-to-width) of at least 2 to 1 with a thin-film layer coated on the sensing side. A probe has a distal end and a proximal end. The proximal end (also refers to probe tip in the application) has a sensing surface coated with a thin layer of analyte-binding molecules.

A “waveguide” refers to a device (e.g., a duct, coaxial cable, or optic fiber) designed to confine and direct the propagation of electromagnetic waves (as light).

The invention uses a high molecular weight conjugate comprising multiple HRP molecules to increase number of enzyme molecules bound to the tip of a BLI biosensor as part of the signal generating step in a sandwich immunoassay format. Horse radish peroxidase (HRP) is often used to increase immunoassay sensitivity in BLI. HRP enzymatic activity generates free radical products, which when reacting with certain substrate can deposit the substrate on the surface of a solid phase matrix used in an assay. In the case of BLI, certain HRP products bind to the surface of the optical layer increase the thickness of the optical layer and cause a shift in the interference pattern. Compared with to conventional HRP reagents, the present invention provides a high molecular weight conjugate comprising multiple HRP molecules, which increases the sensitivity of an immunoassay. This invention further provides unexpected results that the present high molecular weight HRP conjugate significantly increases immunospecific signals without significantly increasing the background caused by non-specific binding, thus the signal-to-noise ratio is improved.

Since the present method measures the HRP enzyme activity by BLI, any non-specific binding by a sample that does not increase HRP conjugate binding, will not cause non-specific signal, which circumvents one of the limitations of BLI. The invention therefore can be applied to crude biological samples that requires high sensitivity detection of an analyte. Consequently, the sensitivity of an immunoassay with BLI is extended by the invention.

### Biosensor Interferometer Systems

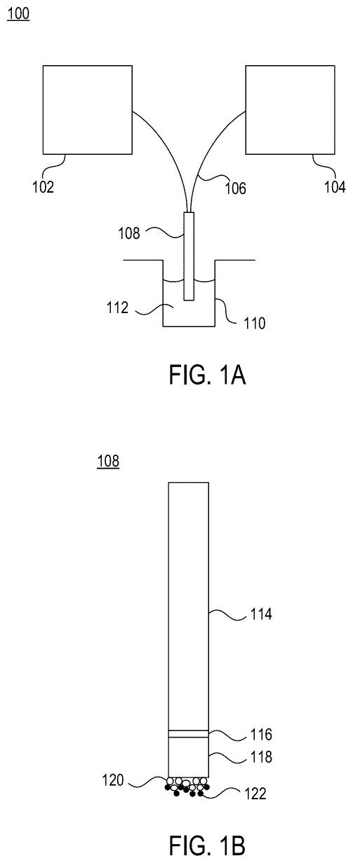

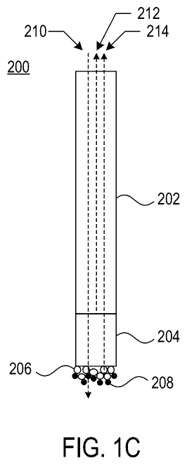

The present invention is suitable for several biosensor interferometer systems. FIGS. 1A-B illustrate one example of such a system. FIG. 1A depicts a biosensor interferometer 100 (or simply “interferometer”) that includes a light source 102, a detector 104, a waveguide 106, and an optical assembly 108 (also referred to as a “probe”). The probe 108 may be connected to the waveguide 106 via a coupling medium.

The light source 102 may emit white light that is guided toward the probe 108 by the waveguide 106. For example, the light source 102 may be a light-emitting diode (LED) that is configured to produce light over a range of at least 50 nanometers (nm), 100 nm, or 150 nm within a given spectrum (e.g., 400 nm or less to 700 nm or greater). Alternatively, the interferometer 100 may employ a plurality of light sources having different characteristic wavelengths, such as LEDs designed to emit light at different wavelengths in the visible range. The same function could be achieved by a single light source with suitable filters for directing light with different wavelengths onto the probe 108.

The detector 104 is preferably a spectrometer, such as an Ocean Optics USB4000, that is capable of recording the spectrum of interfering light received from the probe 108. Alternatively, if the light source 102 operates to direct different wavelengths onto the probe 108, then the detector 104 can be a simple photodetector capable of recording intensity at each wavelength. In another embodiment, the detector 104 can include multiple filters that permit detection of intensity at each of multiple wavelengths.

The waveguide 106 can be configured to transport light emitted by the light source 102 to the probe 108, and then transport light reflected by surfaces within the probe 108 to the detector 104. In some embodiments the waveguide 106 is a bundle of optical fibers (e.g., single-mode fiber optic cables), while in other embodiments the waveguide 106 is a multi-mode fiber optic cable.

As shown in FIG. 1B, the probe 108 includes a monolithic substrate 114, a thin-film layer (also referred to as an “interference layer”), and a biomolecular layer (also referred to as a “biolayer”) comprised of analyte molecules 122 that have bound to analyte-binding molecules 120. The monolithic substrate 114 is comprised of a transparent material through which light can travel. The interference layer is also comprised of a transparent material. When light is shone on the probe 108, the proximal surface of the interference layer may act as a first reflecting surface and the biolayer may act as a second reflecting surface. As further described below, light reflected by the first and second reflecting surfaces may form an interference pattern that can be monitored by the interferometer 100.

The interference layer normally includes multiple layers that are combined in such a manner to improve the detectability of the interference pattern. For example, the interference layer is comprised of a tantalum pentoxide (Ta₂O₅) layer 116 and a silicon dioxide (SiO2) layer 118. The tantalum pentoxide layer 116 may be thin (e.g., on the order of 10-40 nm) since its main purpose is to improve reflectivity at the proximal surface of the interference layer. Meanwhile, the silicon dioxide layer 118 may be comparatively thick (e.g., on the order of 650-900 nm) since its main purpose is to increase the distance between the first and second reflecting surfaces.

To illustrate a simple interferometry test, the probe 108 can be suspended in a well 110 that includes a sample 112. Analyte molecules 122 will bind to the analyte-binding molecules 120 along the distal end of the probe 108 over the course of the diagnostic test, and these binding events will result in an interference pattern that can be observed by the detector 104. The interferometer 100 can monitor the thickness of the biolayer formed along the distal end of the probe 108 by detecting shifts in a phase characteristic of the interference pattern.

FIG. 1C illustrates another biosensor interferometer probe. The probe includes a monolithic substrate that has a first and a second surfaces arranged substantially parallel to one another at opposite ends of the monolithic substrate, an interference layer coated on the second surface of the monolithic substrate, and a layer of analyte-binding molecules coated on the interference layer. The interference layer will generally be comprised of magnesium fluoride (MgF₂). A first interface between the monolithic substrate and the interference layer acts as a first reflecting surface when light is shone on the interferometric sensor, while a second interface between a biolayer formed by analyte molecules in a sample binding to the analyte-binding molecules and a solution containing the sample acts as a second reflecting surface when the light is shone on the probe. As described above, the thickness of the biolayer can be estimated based on the interference pattern of light reflected by the first and second reflecting surfaces.

The probe 200 includes an interference layer 204 that is secured along the distal end of a monolithic substrate 202. Analyte-binding molecules 206 can be deposited along the distal surface of the interference layer 204. Over the course of a biochemical test, a biolayer will form as analyte molecules 208 in a sample bind to the analyte-binding molecules 206.

As shown in FIG. 1C, the monolithic substrate 202 has a proximal surface (also referred to as a “coupling side”) that can be coupled to, for example, a waveguide of an interferometer and a distal surface (also referred to as a “sensing side”) on which additional layers are deposited. Generally, the monolithic substrate 202 has a length of at least 3 millimeters (mm), 5 mm, 10 mm, or 15 mm. In a preferred embodiment, the aspect ratio (length-to-width) of the monolithic substrate 202 is at least 5 to 1. In such embodiments, the monolithic substrate 202 may be said to have a columnar form. The cross section of the monolithic substrate 202 may a circle, oval, square, rectangle, triangle, pentagon, etc. The monolithic substrate 202 preferably has a refractive index that is substantially higher than the refractive index of the interference layer 204, such that the proximal surface of the interference layer 204 effectively reflects light directed onto the probe 200. The preferred refractive index of the monolithic substrate may be higher than 1.5, 1.8, or 2.0. Accordingly, the monolithic substrate 202 may be comprised of a high-refractive-index material such as glass (refractive index of 2.0) rather than a low-refractive-index material such as quartz (refractive index of 1.46) or plastic (refractive index of 1.32-1.49).

The interference layer 204 is comprised of at least one transparent material that is coated on the distal surface of the monolithic substrate 202. These transparent material(s) are deposited on the distal surface of the monolithic substrate 202 in the form of thin films ranging in thickness from fractions of a nanometer (e.g., a monolayer) to several micrometers. The interference layer 204 may have a thickness of at least 500 nm, 700 nm, or 900 nm. An exemplary thickness is between 500-5,000 nm (and preferably 800-1,200 nm). Here, for example, the interference layer 204 has a thickness of approximately 900-1,000 nm, or 940 nm.

In contrast to conventional probes, the interference layer 204 has a substantially similar refractive index as the biolayer. This ensures that the reflection from the distal end of the probe 200 is predominantly due to the analyte molecules 208 rather than the interface between the interference layer 204 and the analyte-binding molecules 206. In some embodiments, the interference layer 204 is comprised of magnesium fluoride (MgF₂), while in other embodiments the interference layer 204 is comprised of potassium fluoride (KF), lithium fluoride (LiF), sodium fluoride (NaF), lithium calcium aluminum fluoride (LiCaAlF₆), sodium aluminum fluoride (Na₃AlF₆), strontium fluoride (SrF₂), aluminum fluoride (AlF₃), sulphur hexafluoride (SF₆), etc. Magnesium fluoride has a refractive index of 1.38, which is substantially identical to the refractive index of the biolayer formed along the distal end of the probe 200. For comparison, the interference layer of conventional probes is normally comprised of silicon dioxide, and the refractive index of silicon dioxide is approximately 1.4-1.5 in the visible range. Because the interference layer 204 and biolayer have similar refractive indexes, light will experience minimal scattering as it travels from the interference layer 204 into the biolayer and then returns from the biolayer into the interference layer 204.

In one embodiment, the probe 200 includes an adhesion layer that is deposited along the distal surface of the interference layer 204 affixed to the monolithic substrate 202. The adhesion layer may comprise a material that promotes adhesion of the analyte-binding molecules 206. One example of such a material is silicon dioxide. The adhesion layer is generally very thin in comparison to the interference layer 204, so its impact on light traveling toward, or returning from, the biolayer will be minimal. For example, the adhesion layer 310 may have a thickness of approximately 3-10 nm, while the interference layer 304 may have a thickness of approximately 800-1,000 nm. The biolayer formed by the analyte-binding molecules 306 and analyte molecules 308 will normally have a thickness of several nm.

When light is shone on the probe 200, the proximal surface of the interference layer 204 may act as a first reflecting surface and the distal surface of the biolayer may act as a second reflecting surface. The presence, concentration, or binding rate of analyte molecules 208 to the probe 200 can be estimated based on the interference of beams of light reflected by these two reflecting surfaces. As analyte molecules 208 attach to (or detach from) the analyte-binding molecules 206, the distance between the first and second reflecting surfaces will change. Because the dimensions of all other components in the probe 200 remain the same, the interference pattern formed by the light reflected by the first and second reflecting surfaces is phase shifted in accordance with changes in biolayer thickness due to binding events.

In operation, an incident light signal 210 emitted by a light source is transported through the monolithic substrate 202 toward the biolayer. Within the probe 200, light will be reflected at the first reflecting surface resulting in a first reflected light signal 212. Light will also be reflected at the second reflecting surface resulting in a second reflected light signal 214. The second reflecting surface initially corresponds to the interface between the analyte-binding molecules 206 and the sample in which the probe 200 is immersed. As binding occurs during the biochemical test, the second reflecting surface becomes the interface between the analyte molecules 208 and the sample.

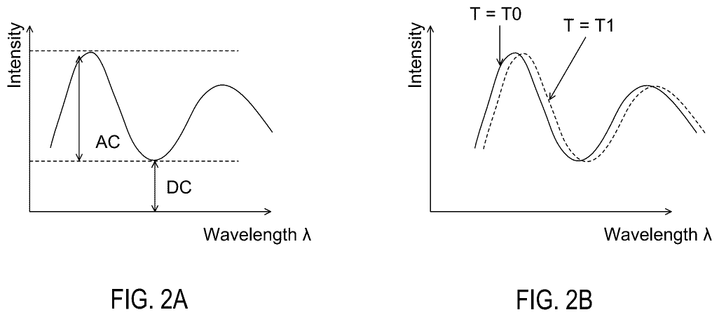

The two light signals reflected from boundaries between first and second refracting surfaces generate a spectral interference pattern, as shown in FIG. 2A. When analyte molecules bind to the analyte-binding molecules on the distal surface of the interference layer, the optical path of the second reflected light signal lengthens. As a result, the spectral interference pattern shifts from T0 to T1 as shown in FIG. 2B. By measuring the phase shift after a period of time, the association of an analyte molecule to an analyte-binding molecule immobilized on the distal surface of the interference layer 204 can be used to calculate analyte concentration in the sample. By measuring the phase shift continuously in real time, a kinetic binding curve can be plotted as the amount of shift versus the time. The association rate of an analyte molecule to an analyte-binding molecule immobilized on the distal surface of the interference layer can be used to calculate analyte concentration in the sample. Hence, the measure of the phase shift is the detection principle of a thin-film interferometer.

### Methods for Determining Analyte Concentration

The present invention is directed to a method of determining an analyte concentration in a sample. The method comprises the steps of: (a) obtaining a probe having a first antibody against the analyte immobilized on the tip of the probe, wherein the diameter of the tip surface is ≤5 mm; (b) dipping the probe tip into a sample solution comprising the analyte to capture the analyte at the probe tip; (c) dipping the probe in a reagent solution comprising a second antibody against the analyte, wherein the second antibody is labelled with biotin; (d) dipping the probe in a conjugate solution having a conjugate comprising (i) streptavidin, HRP and a polymer, or (ii) streptavidin and crosslinked HRP, or (iii) crosslinked streptavidin and HRP, to bind the conjugate to the probe, wherein the conjugate has a molecular weight at least 400,000 daltons, and comprises at least 4 HRPs; (e) dipping the probe in a HRP substrate solution to bind the substrate to the HRP in the conjugate for a period of time; and (f) determining the analyte concentration in the sample by measuring the wavelength shift due to light interference, and quantitating the wavelength shift against a calibration curve to determine the analyte concentration.

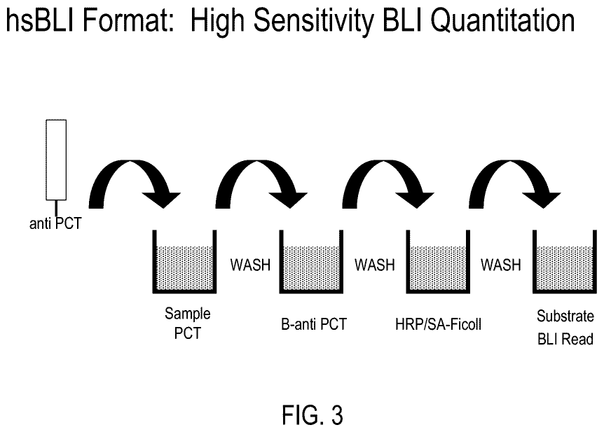

FIG. 3 depicts one embodiment of a high sensitivity BLI immunoassay for procalcitonin (PCT) in a sandwich assay format. A BLI probe coated with an antibody to PCT on the tip is immersed in a sample containing PCT. After a wash sequence, the probe is transferred to a biotin labeled anti-PCT reagent to form a sandwich immune complex with PCT captured on the probe. After another wash sequence, the probe is transferred to a streptavidin/HRP-FICOLL® (copolymers of sucrose and epichlorohydrin) reagent that binds to biotin on the probe surface. Following a wash sequence, the probe is placed in a HRP substrate solution, and the enzyme activity bound on the probe is monitored by a BLI instrument, for a period of time, e.g., about 30 seconds. A HRP substrate is selected based on the binding of its enzymatic product to the surface of the probe to increase the thickness of the biolayer, which then produces a detectable shift in the BLI interference pattern.

In step (a) of the present method, a probe that has a small tip for binding an analyte is obtained. The tip has a smaller surface area with a diameter ≤5 mm, preferably ≤2 mm or ≤1 mm. The small surface of the probe tip endows it with several advantages. In solid phase immunoassays, a probe having a small surface area is advantageous because it has less non-specific binding and thus produces a lower background signal. Further, the reagent or sample carry over on the probe tip is extremely small due to the small surface area of the tip. This feature makes the probe tip easy to wash and results in negligible contamination in the wash solution since the wash solution has a larger volume. Another aspect of the small surface area of the probe tip is that it has small binding capacity. Consequently, when the probe tip is immersed in a reagent solution, the binding of the reagent does not consume a significant amount of the reagent. The reagent concentration is effectively unchanged. Negligible contamination of the wash solution and small consumption of the reagents enable the reagents and the wash solution to be re-used many times, for example, 3-10 times, 3-15 times, or 3-20 times.

Methods to immobilize a protein such as an antibody to a solid phase (the sensing surface of the probe tip) are common in immunochemistry. An antibody can bind directly to the solid phase through adsorption or it can bind indirectly to the solid phase through a binding pair. For example, the probe surface can be coated with a first member of the binding pair (e.g. anti-hapten), and an an antibody labelled with a second member of the binding pair (e.g., hapten) is immobilized on the probe through the biotin-streptavidin binding.

In step (b) of the method, the probe tip is dipped in a sample solution comprising an analyte for a period of time to capture the analyte on the probe in a defined binding condition. In one embodiment, the binding time is from 1 minute or 2 minutes to 30 minutes or 1 hour. For example, the binding time is from 2-10 minutes.

After the binding step, the probe is optionally dipped in a wash solution comprising an aqueous solution preferably having pH of 6.0-8.5 for a period of time (e.g., 5 seconds to 5 minutes, 10 seconds to 2 minutes, or 15 seconds to 1 minute). The aqueous solution can be water or a buffer having pH between 6.0 to 8.5. Preferably, the aqueous solution contains 1-10 mM or 1-100 mM of phosphate buffer, tris buffer, citrate buffer or other buffer suitable for pH between 6.0-8.5. The probe is washed 1-3 times.

In step (c), the probe is dipped in a reagent solution comprising biotin-labelled second antibody against the analyte for a period of time (e.g., 30 seconds to 5 minutes), wherein the second antibody binds to the analyte.

The probe is then optionally washed in a wash solution as described in step (b).

In step (d), the probe is dipped in a conjugate solution comprising a high molecular weight conjugate comprising streptavidin and horse radish peroxidase (HRP) for a period of time (e.g., 10 seconds to 5 minutes, 15 seconds to 1 minute, or 20 seconds to 2 minutes), to bind the conjugate to the probe.

In general, the high molecular weight conjugate has a molecular weight of at least 200,000, or at least 400,000, or at least 500,000, at least 1 million, or at least 2 million Daltons. For example, the conjugate has a molecular weight of 200,000 to 10,000,000 Daltons, 500,000 to 10 million Daltons, 1 million to 10 million Daltons, or 2 million to 10 million Daltons.

In one embodiment, the high molecular weight conjugate comprises streptavidin and crosslinked HRP, wherein the conjugate comprises at least 2, or at least 4, or at least 5, or at least 10 HRPs.

In one embodiment, the high molecular weight conjugate comprises streptavidin and HRP, where streptavidin and HRP are crosslinked together. The conjugate comprises at least 2, or at least 4, at least 5, or at least 10 HRPs, and the molar ratio of streptavidin to HRP is between about 1:2 to about 2:1, or about 1:1.5 to about 1.5 to 1, e.g., about 1:1.

In a preferred embodiment, the high molecular weight conjugate comprises streptavidin, HRP and a polymer. In the conjugate, streptavidin and HRP may bind to the polymer. The polymer in general has a molecular weight of 200,000 to 10 million Daltons, or 500,000 to 10 million Daltons, or 1 million to 10 million Daltons. The polymer can be a polysaccharide (e.g., dextran, amylose, polysucrose), a dendrimer, or a polyethylene glycol. In one preferred embodiment, the polymer is FICOLL® (copolymers of sucrose and epichlorohydrin). The conjugate comprises at least 2, or at least 4, at least 5, or at least 10 HRPs, and the molar ratio of streptavidin to HRP is between about 1:2 to about 2:1, or about 1:1.5 to about 1.5 to 1, e.g., about 1:1.

After binding, the probe is then washed in a wash solution as described in step (c) for 1-3 times.

In step (e), the probe is dipped in a HRP substrate solution to bind the substrate to the HRP in the conjugate for a period of time (e.g., 30 seconds to 5 minutes).

When the product of the HRP/substrate reaction binds to the surface of the optical layer, the optical layer increases its thickness and produces a nanometer wavelength shift of light interference, and the wavelength shift is proportional to the amount of HRP bound on the probe. Any HRP substrate whose product binds to solid phase surfaces is suitable for the present invention. A precipitating substrate is preferred for BLI assay. Suitable substrates include 3,3′-diaminobenzidine tetrahydrochloride (DAB), 3,3′,5,5′-tetramethylbenzidine (TMB), benzamidine, chloronaphthol, nitro-blue tetrazolium chloride and 5-bromo-4-chloro-3′-indolyphosphate p-toluidine salt. Preferred substrates are chloronapthol, benzamidine, and tetramethylbenzidine, and chloronapthol is more preferred.

In step (f), the analyte concentration in the sample is determined by measuring the wavelength shift of light interference, and the wavelength shift is quantitated against a calibration curve having wavelength shift plotted against the analyte concentration to determine the analyte concentration. The phase shift can be monitored either kinetically or determined by the difference between starting time point (T0) and end time point (T1) (see FIG. 2B).

In the above binding and washing steps, the reaction is optionally accelerated by agitating or mixing the solution in the vessel. For example, a flow such as a lateral flow or an orbital flow of the solution across the probe tip can be induced in one or more reaction vessels, including sample vessel, reagent vessel, wash vessels, and conjugate vessel, to accelerates the binding reactions, disassociation. For example, the reaction vessels can be mounted on an orbital shaker and the orbital shaker is rotated at a speed at least 50 rpm, preferably at least 200 rpm or at least 500 rpm, such as 50-200 or 500-1,500 rpm. Additionally, the probe tip can be moved up and down and perpendicular to the plane of the orbital flow, at a speed of 0.01 to 10 mm/second, in order to induce additional mixing of the solution above and below the probe tip.

The assay protocol as shown in FIG. 3 is for a sandwich assay format. A competitive assay format can also be designed using the high molecule weight streptavidin/HRP-FICOLL® conjugate.

### High Molecular Weight Conjugate

The present invention is also directed to a conjugate comprises streptavidin, HRP and crosslinked FICOLL®. The crosslinked FICOLL® has a molecular weight of at least 500,000 daltons, and preferably at least 1 million, or 2 million, or 5 million, or 10 million Daltons. The conjugate has at least 2 streptavidin and at least 2 HRPs, and preferably has at least 4 streptavidin and at least 4 HRPs. In one embodiment, the crosslinked FICOLL® has a molecular weight of 500,000-10 million Daltons, or 1-10 million Daltons, and each crosslinked FICOLL® binds at least 2 HRPs and at least 2 streptavidins. In one embodiment, the crosslinked FICOLL® has a molecular weight of at least 2 million Daltons (e.g., 1-10 million Daltons), and each crosslinked FICOLL® binds at least 4 or 5 HRPs and at least 4 or 5 streptavidin. In one embodiment, the crosslinked FICOLL® has a molecular weight of at least 2 million Daltons or at least 5 million Daltons (e.g., 2-10 million Daltons or 5-10 million Daltons), and each crosslinked FICOLL® binds at least 10 HRPs and at least 10 streptavidins.

In one embodiment, the conjugate has a molar ratio of FICOLL®: streptavidin: HRP of 1:2-10:2-10. In another embodiment, the conjugate has a molar ratio of FICOLL®: streptavidin: HRP of 1:3-7:3-7. For example, each crosslinked FICOLL® molecules binds at least 4-10 HRP and 4-10 streptavidin.

### Probes

The present invention is also directed to a probe comprising a monolithic substrate, a thin-film layer, and a biomolecular layer immobilized on the tip of the probe, wherein the biomolecular layer comprises analyte molecules, analyte-binding molecules, HRP, and chloronapthol, wherein the diameter of the tip surface is ≤5 mm.

The invention is illustrated further by the following examples that are not to be construed as limiting the invention in scope to the specific procedures described in them.

### EXAMPLES

### Materials

Recombinant streptavidin was obtained from IBA. Horseradish peroxidase (HRP) RZ=1 was obtained from Santa Cruz BioTech. SMCC, succinimidyl 4-(N-maleimidomethyl) cyclohexane-1-carboxylate, DMF, anhydrous dimethylformamide were obtained from Thermo Scientific without further purification before usage. Cy5-NHS, Cyanine 5 monosuccinimidyl ester was ordered from ATT Bioquest. N-acetyl-1-cysteine and n-ethylmaleimde were purchased from MP Biomedicals and Thermo Scientific.

### Instrumentation

UV/VIS measurements were done by Spectrophotometer (Thermo BioMate 3S). Centrifugation was done by Centrifuge (Beckman Allegra 6R). Protein was desalted by Zebra Spin columns 2 ml size, 7K MWCO (Thermo Scientific). Bioconjugates were purified with Cytiva CL-4B Sepharose medium custom packed in XK 16×400 column connected to Cytiva AKTA Go Purification System.

Gator®Prime system (Gator Bio, Inc.) was used for BLI measurement.

### Example 1. Preparation of Crosslinked-FICOLL® 400-SPDP

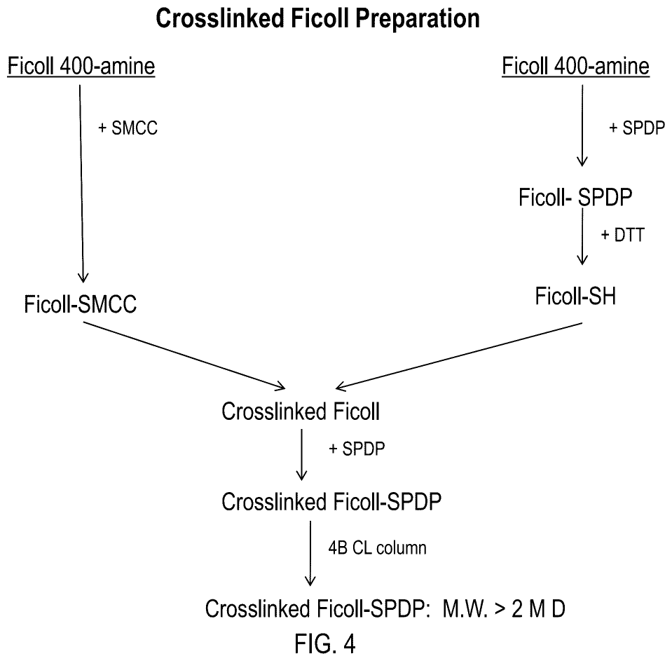

Crosslinked-FICOLL 400-SPDP (succinimydyl 6-[3-[2-pyridyldithio]-proprionamido]hexanoate, Invitrogen) was prepared according to Example 1 of US 2011/0312105. FIG. 4 shows a flow chart of its preparation.

### Example 2. Preparation of Cyanine 5 and Maleimide Labelled Streptavidin

10 mg of Streptavidin (molecular weight about 60,000 Daltons) was dissolved in 500 μl PBS buffer to make it as 20 mg/ml. 50 μl of 0.5M carbonate buffer was added to the Streptavidin solution. 30 μl (MCR=1) of 5 mg/ml of cyanine 5 (Cy5) monosuccinimidyl ester in anhydrous dimethylformamide (DMF) was added to above Streptavidin solution and let it react at room temperature for 1 hour. The Cy5 in this case served as a tracer enabling quantification of the streptavidin content in the subsequently prepared Cy5-streptavidin/HRP-crosslinked FICOLL® conjugate. 10 mg/ml SMCC, succinimidyl 4-(N-maleimidomethyl) cyclohexane-1-carboxylate in anhydrous dimethylformamide (DMF) was freshly prepared and 52 μl (MCR=8.7) of this was added to Cy5 labelled Streptavidin aforementioned. The SMCC labelling reaction was run at room temperature for another 1 hour. 2 ml size Zeba Spin Desalting column (7K MWCO) prepared following Thermo Scientific Instruction Guide. Cy5/SMCC dual labeled streptavidin reaction mixture was loaded to the column and centrifuge at 1000×g for 2 min, the flow-through that contains sample was collected. The concentration of Streptavidin and Cy5 labelling ratio were estimated by 280 and 655 nm absorption by Spectrophotometer. Streptavidin is labeled with Cy5 so that the labelled streptavidin can be monitored throughout the conjugation step.

### Example 3. Preparation of Maleimide Labelled Horseradish Peroxidase

12 mg of Horseradish peroxidase (HRP), molecular weight about 40,000 Daltons, was dissolved in 600 μl 1×PBS buffer, followed with 60 μl of 0.5M Carbonate buffer addition. 72 μl of freshly prepared 10 mg/ml SMCC, succinimidyl 4-(N-maleimidomethyl) cyclohexane-1-carboxylate in anhydrous dimethylformamide (DMF) was added to the HRP solution to make up MCR=7.2 of SMCC vs HRP molar ratio. After 1 hour of room temperature reaction, the 2 ml Zeba Spin Desalting column (7K MWCO) was prepared by Thermo Scientific Instruction Guide. Reaction mixture of maleimide labelled HRP was loaded on the top of the spin column and centrifuge at 1000×g for 2 min. The purified sample was collected and the amount of maleimide labelled HRP was estimated by 280 nm absorption by Spectrophotometer.

### Example 4. Preparation of Cy5-Streptavidin, Horseradish Peroxidase, Crosslinked FICOLL® Conjugates

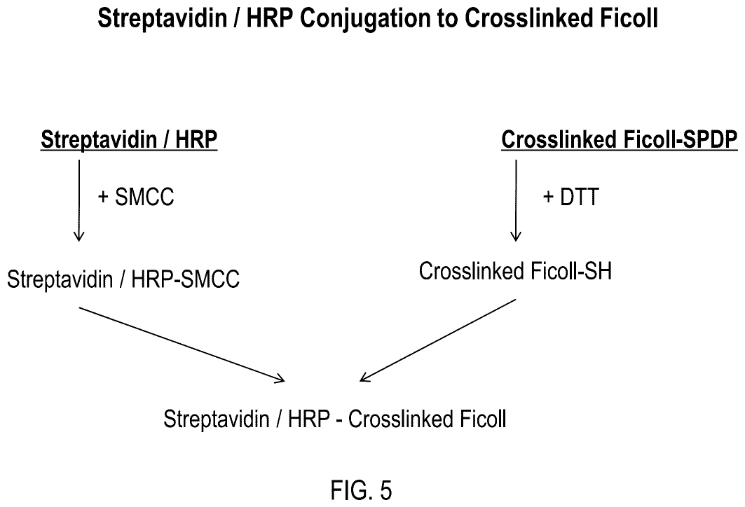

The flow chart of preparing streptavidin/HRP-Crosslinked FICOLL® is depicted on FIG. 5. Crosslinked FICOLL®-SPDP, (succinimidyl 3-(2-pyridyldithio) propionate), reacted with DTT, (Dithiothreitol) 38 mg/ml with 1:500 fold molar excess for 1 hour at room temperature. At the end of reaction, the mixture was loaded to a 2ml size Zebra Spin Desalting column (7K MWCO) which was prepared by Thermo Scientific Instruction Guide. The mercapto labelled crosslinked FICOLL® was collected as the flow-through of the Zeba Spin column by centrifuge at 1000×g for 2 min. This sample was reacted with the cyanine 5/maleimide labelled streptavidin (Example 2) and maleimide labelled horseradish peroxidase (Example 3) in the Table 1.

TABLE 1
Starting Ratio of Regents in Reaction Mixture

| Crosslinked | Cy5-Streptavidin | | |
|---|---|---|---|
| FICOLL ® | (SA)-SMCC | HRP-SMCC | Molar ratio |
| (mg) | (mg) | (mg) | (FICOLL ®:SA:HRP) |
| 1 | 1.1 | 0.8 | 1:4:5 |

The Coupling of Crosslinked FICOLL® mercaptan (1 mg) with maleimide-streptavidin (1.1 mg) and maleimide-horseradish peroxidase (0.8 mg) proceeded at room temperature for 4 hours, then quenched with n-acetyl-1-cysteine 16 mg/ml in 1 ×PBS (6.25 μl per mg of Streptavidin usage) and n-ethylmaleimde 32 mg/ml in 1×PBS (10 μl per mg of Streptavidin usage) sequentially. The quenched reaction mixture was purified with CL-4B column with AKTA Go purification system in 1 ml/min flow rate (1×PBS mobile phase). The first eluted peak was the desired product. About 90% of streptavidin and HRP are conjugated to the crosslinked FICOLL®.

### Example 5. Preparation of Anti-Fluorescein-Crosslinked FICOLL® Conjugates

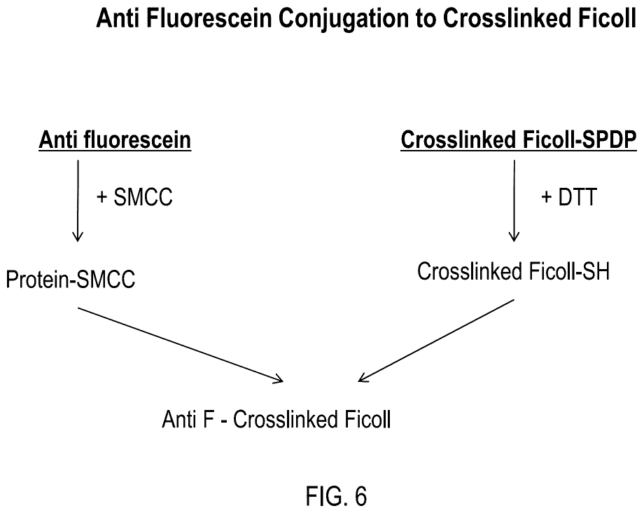

FIG. 6 presents the flow chart for the preparation of anti-fluorescein-crosslinked FICOLL®. Anti-fluorescein (Jackson ImmunoResearch) at 1.5 mg/ml in 1 ml PBS was mixed with 1.9 μl SMCC at 5 mg/ml DMF and reacted for 1 hour at room temperature followed by purification on a PD 10 column.

The thiols on crosslinked FICOLL®-SPDP were deprotected by adding 30 μl DTT at 38 mg/ml to 0.7 mg crosslinked FICOLL® 400-SPDP in 1 ml PBS and reacting for 1 hour at room temperature followed by a PD 10 column to purify the crosslinked FICOLL®. The anti-fluorescein-SMCC was mixed with crosslinked FICOLL®-SH and reacted overnight at room temperature. 10 μl NEM (Aldrich) at 12.5 mg/ml was then added and reacted for ½ hour at room temperature. The conjugate was then purified on a Sepharose 4B CL column.

### Example 6: Preparation of Streptavidin-HRP Conjugate (Comparison Purpose)

Streptavidin (IBA 2-0203-100) 6.3 mg in 1.5 ml PBS pH 7.4 reacted with 28 μl SPDP (succinimidyl 3-(2-pyridyldithio) propionate, Invitrogen 51531) at 10 mg/ml of DMF and allowed to react for 1 hour at room temperature. The material was then purified on a Sephadex G25 column. To generate free thiols, the streptavidin-SPDP 4.5 mg in 1.5 ml PBD S reacted with 30 μl dithiolthreitol 38 mg/ml PBS and reacted for 30 minutes at room temperature, followed by purification with a Sephadex G25 column. HRP (Santa Cruz Bio H0621) 3.7 mg in 1 ml PBS reacted with 21 μl SMCC 10 mg/ml DMF, followed by purification on a Sephadex G25 column. Streptavidin-SH and HRP-SMCC were mixed, reacting overnight at 4C. The conjugate was then purified on a Sepharose 6B CL column. The column was pre-calibrated with molecular weight markers, streptavidin, IgG and beta-galactosidase. The streptavidin-HRP conjugate with an estimated molecular weight of 200,000 Daltons was collected.

### Example 7. Preparation of Reagents and Anti-PCT Probe for PCT Assay

Quartz probes, 1 mm diameter and 2 cm in length, with BLI optical layer at the distal tip were coated with aminopropylsilane using a chemical vapor deposition process (Yield Engineering Systems, 1224P) following manufacturer's protocol. The probe tip was then immersed in a solution of murine monoclonal anti-fluorescein-crosslinked FICOLL® (from Example 5) at 10 μg/ml in PBS at pH 7.4. After allowing the antibody to adsorb to the probe for 15 minutes, the probe tip was washed in PBS.

The probe tip was then immersed in a solution containing fluorescein-labeled murine monoclonal anti-procalcitonin PCT (Hytest) at 10 μg/ml. The antibody was fluorescein labeled by a standard method. After 10 minutes, the probe was washed in PBS. Probes were then immersed in a solution of 15% sucrose followed by drying the probes at 37° C. for 20 minutes. The polyclonal anti PCT (Hytest) was biotinylated by a standard method.

### Example 8: High Sensitivity BLI Assay for PCT

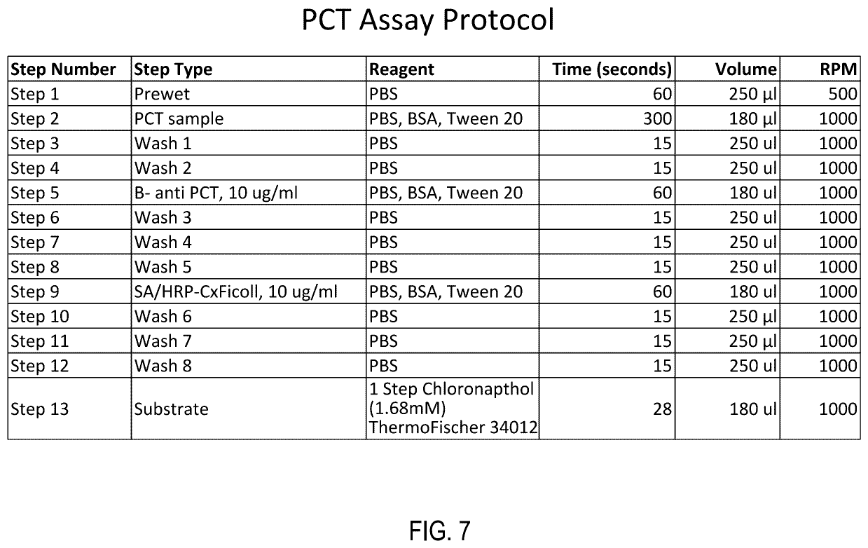

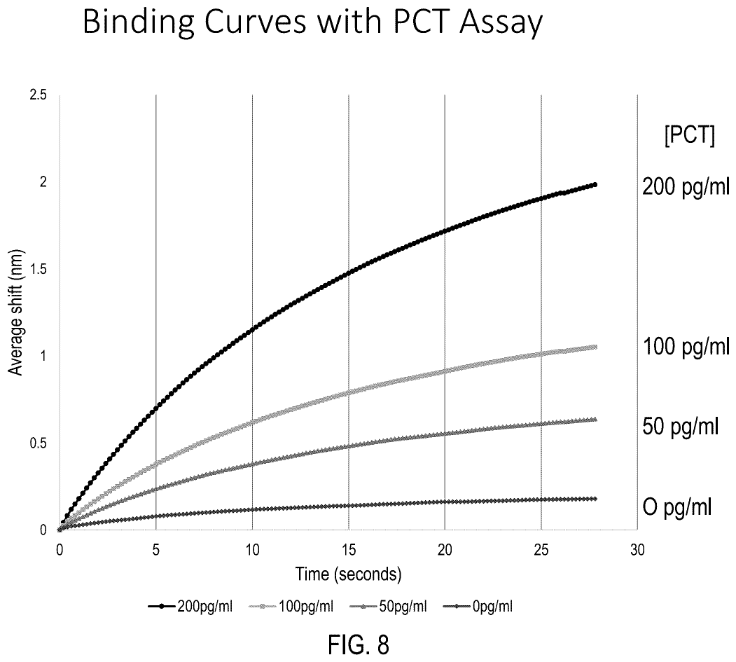

FIG. 7 presents a table of the protocol, reagents, timing steps, volumes, and orbital rpm in performing the PCT assay. FIG. 8 shows the results of the BLI measurements at step 13 monitoring the probe bound HRP enzyme activity when the probe tip is immersed in the substrate reagent. Suitable substrates include 3,3′-diaminobenzidine tetrahydrochloride (DAB), 3,3′,5,5′-tetramethylbenzidine (TMB), benzamidine, chloronaphthol, nitro-blue tetrazolium chloride and 5-bromo-4-chloro-3′-indolyphosphate p-toluidine salt. Preferred substrates are chloronapthol, benzamidine, and tetramethylbenzidine, and chloronapthol is more preferred. PCT samples at 0, 50, 100, and 200 pg/ml produced a dose response with different nanometer shifts.

### Example 9: Comparison of Streptavidin-HRP and Streptavidin/HRP-Crosslinked FICOLL®

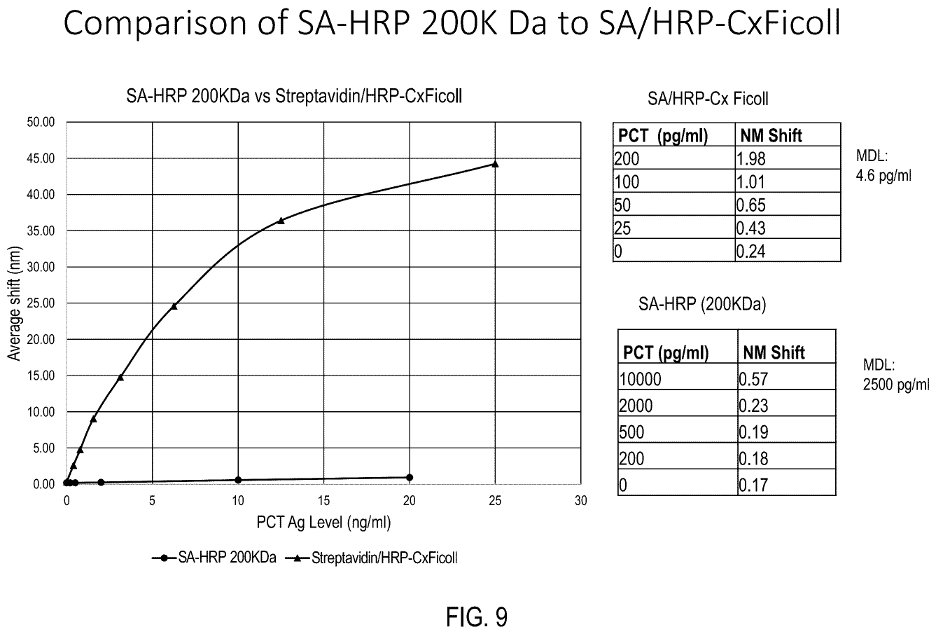

A comparison with different streptavidin-HRP conjugates was made in a high sensitivity BLI format. A conjugate with a direct linkage between streptavidin and HRP as described in Example 6 with a molecular weight of about 200,000 Daltons, and a streptavidin/HRP-crosslinked FICOLL® conjugate as described in Example 4 with a molecular weight estimated to be several million Daltons were used in the PCT assay as described in Example 8. Both conjugates were loaded in the assay at Step 9 at 10 μg/ml. FIG. 9 presents PCT curves using both conjugates, where the streptavidin/HRP crosslinked (Cx) FICOLL® generated vastly higher nanometer shift signals. The PCT assay with streptavidin/HRP-CxFicoll was about 500 times more sensitive than the streptavidin-HRP, minimum detection limits (MDL) were respectively 4.6 pg/ml vs 2500 pg/ml. MDL was defined as PCT concentration at 2 times the standard deviation of negative samples. The streptavidin/HRP-Cx FICOLL® reagents increased the specific PCT signals without a significant increase in background with negative samples.

### Example 10: Comparing Different Streptavidin-HRP Conjugates in PCT Assay

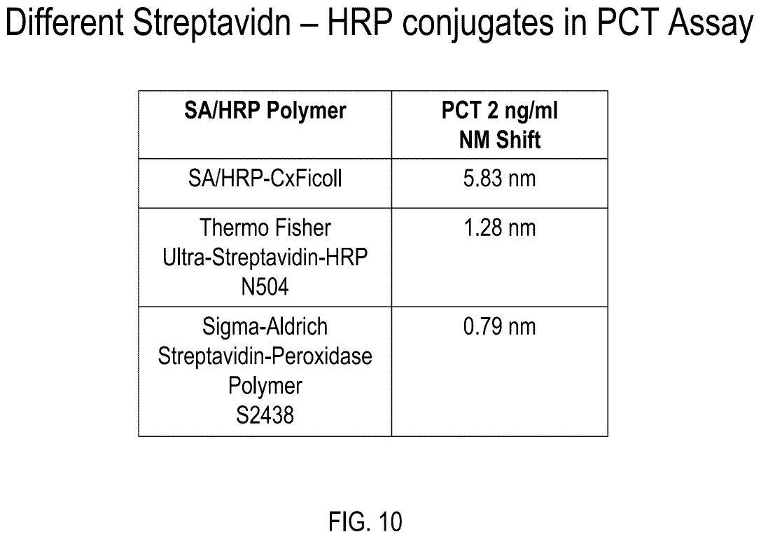

In FIG. 10, results comparing the streptavidin/HR-Cx FICOLL® with two commercial streptavidin-HRP reagents in the PCT assay at 2 ng/ml of PCT with the protocol of Example 8, using different HRP reagents at Step 9. All three HRP reagents produced some wavelength (nm) shift in the BLI assay, and the SA/HRP-crosslinked FICOLL produced the highest wavelength (nm) shift (5.83 nm vs. 1.28 nm or 0.79 nm).

### Example 11: Correlation with Clinical Samples

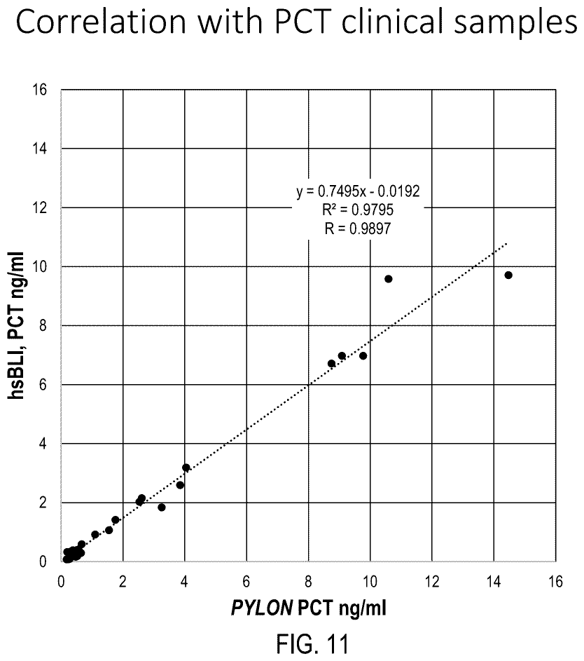

Plasma clinical PCT samples were analyzed by high sensitivity BLI and Pylon 3D instrument (ET Healthcare Inc), a commercial clinical immunoanlyzer using fluorescence detection. Samples were analyzed by BLI following the protocol in Example 8 and by Pylon 3D following the manufacturer's recommended protocol. FIG. 11 shows a high correlation (R=0.98) between the methods, indicating that high sensitivity BLI can be used in applications with crude biological samples that requires high sensitivity and accurate quantitation.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A method of detecting an analyte in a sample, comprising the steps of:
(a) obtaining a probe having a first antibody against the analyte immobilized on the tip of the probe, wherein the diameter of the tip surface is ≤5 mm;
(b) dipping the probe tip into a sample solution comprising the analyte to capture the analyte at the probe tip;
(c) dipping the probe in a reagent solution comprising a second antibody against the analyte, wherein the second antibody is labelled with biotin;
(d) dipping the probe in a conjugate solution having a conjugate comprising (i) streptavidin, HRP and a polymer, or (ii) streptavidin and crosslinked HRP, or (iii) crosslinked streptavidin and HRP, to bind the conjugate to the probe, wherein the conjugate has a molecular weight at least 400,000 Daltons, and comprises at least 4 HRPs;
(e) dipping the probe in a HRP substrate solution to bind the substrate to the HRP in the conjugate for a period of time; and
(f) determining the analyte concentration in the sample by measuring the wavelength shift due to light interference, and quantitating the wavelength shift against a calibration curve to determine the analyte concentration.

2. The method of claim 1, wherein the conjugate comprises (i) streptavidin, HRP, and the polymer, and the polymer has a molecular weight at least 1 million Daltons.

3. The method of claim 1, wherein the conjugate comprises (i) streptavidin, HRP, and the polymer, and the polymer has a molecular weight at least 2 million Daltons.

4. The method of claim 1, wherein the conjugate comprises (i) streptavidin, HRP, and the polymer, and the polymer is crosslinked copolymers of sucrose and epichlorohydrin.

5. The method of claim 4, wherein the polymer has a molecular weight over 1 million Daltons.

6. The method of claim 4, wherein the polymer has a molecular weight over 2 million Daltons.

7. The method of claim 1, wherein the substrate is chloronapthol, benzamidine, or tetramethylbenzidine.

8. The method of claim 7, wherein the substrate is chloronapthol.

9. The method of claim 2, wherein the substrate is chloronapthol, benzamidine, or tetramethylbenzidine.

10. The method of claim 3, wherein the substrate is chloronapthol, benzamidine, or tetramethylbenzidine.

11. The method of claim 4, wherein the substrate is chloronapthol, benzamidine, or tetramethylbenzidine.

12. The method of claim 5, wherein the substrate is chloronapthol, benzamidine, or tetramethylbenzidine.

13. A conjugate comprises streptavidin, HRP and crosslinked copolymers of sucrose and epichlorohydrin, wherein the crosslinked copolymers of sucrose and epichlorohydrin has a molecular weight of at least 1 million Daltons, and each crosslinked copolymer of sucrose and epichlorohydrin binds at least 2 HRP and at least 2 streptavidin.

14. The conjugate of claim 13, wherein the crosslinked copolymers of sucrose and epichlorohydrin has a molecular weight of at least 2 million Daltons, and each crosslinked copolymer binds at least 4 HRP and at least 4 streptavidin.

15. The conjugate of claim 13, wherein the conjugate has a molar ratio of the crosslinked copolyymer: streptavidin: HRP of 1:3-7:3-7.

16. The conjugate of claim 14, wherein the conjugate has a molar ratio of the crosslinked copolyymer: streptavidin: HRP of 1:3-7:3-7.
